(19) United States
(12) Patent Application Publication
(10) Pub. No.: US 2008/0038719 A1
(43) Pub. Date: Feb. 14, 2008

(54) Nucleic Acid Composite Materials Made Sensors For The Analysis Of Nucleic Acid Modifying Factors

(76) Inventors: Gregoire Lagger, Geneva (CH); Philippe Tacchini, Geneva (CH); Hubert H. Girault, Lausanne (CH)

Correspondence Address: KNOBBE MARTENS OLSON & BEAR LLP, 2040 MAIN STREET, FOURTEENTH FLOOR, IRVINE, CA, 92614, US

(21) Appl. No.: 10/585,180

(22) PCT Filed: Dec. 30, 2003
(86) PCT No.: PCT/EP03/14973

§ 371 Date: Jun. 20, 2007

(51) Int. Cl. C12Q 1/68; C07H 21/00; C12M 1/00

(52) U.S. Cl. 435/6; 435/287.2; 536/23.1

(57) ABSTRACT

The present invention relates to a method for fabricating a nucleic acid composite material, and to a sensor fabricated with this composite material. This composite material sensor can be used as the working electrode in a conventional electrochemical system, for the measurement of any nucleic acid modifying factors. Protective and/or damaging effects of oxidants/anti-oxidants present in the solution can then be analyzed based on their action on nucleic acids.

The present invention relates to a method for fabricating a nucleic acid composite material, and to a sensor fabricated with this composite material. This composite material sensor may be used as the working electrode in a conventional electrochemical system for the measurement of nucleic acid modifying factors. Protective and/or damaging effects of oxidants/anti-oxidants present in the solution may then be analyzed based on their action on nucleic acids.

In the recent past a variety of biosensors have been developed for detecting biological material, such as diverse cellular components, e.g. nucleic acids or proteins, or even entire micro-organisms. In general, such sensors are required to exhibit a high specificity for the particular entity to be detected or determined and a high sensitivity, so that even a low number/amount of the specific entity may be detected.

Electrochemical DNA biosensors with an immobilized layer of the DNA discriminator are known as simple and sensitive bioanalytical devices for the detection of DNA damage. These sensors include oxidized pyrolytic graphite electrodes onto which films of poly(dimethyldiallylammonium chloride) (PDDA) cations and (ds)-DNA have been deposited. Solid phase electrodes in a combination with sensitive detection techniques became of interest as modern nucleic acid probes. However, the fabrication of these sensors is cumbersome and requires many steps, such as electrode polishing and film assembly.

U.S. Pat. No. 6,638,415 describes a device for measuring the level of oxidant or anti-oxidant analytes in a fluid sample. The device consists of a disposable electrochemical cell containing a reagent capable of undergoing a redox reaction with the analyte. Heat may be applied by a resistive heating element or by an exothermic material contained within the cell, in case slow reacting analytes are to be used.

In addition, U.S. Pat. No. 6,063,259 discloses a thick film sensing apparatus for nucleic acid determination and testing using potentiometric stripping analysis including nucleic acid analysis, based on modified (DNA coated) screen-printed electrode.

An object of the present invention resides in obviating the shortcomings of prior art and to provide a new means for measuring nucleic acids (DNA) modifications.

This objective has been achieved by providing a composite material to be used as a working electrode in a cell, which is obtainable by the steps of (i) providing conductive particles; (ii) optionally pre-treating the particles by physical or chemical means; (iii) mixing nucleic acid material with conductive particles; (iv) depositing the composite material onto a carrier or molding the composite material to be used as a working electrode; and (v) drying the formulation.

In particular, the present invention provides a composite material formulation and fabrication method for the preparation of an electrochemical sensor using nucleic acids as target/test probes which may be used for the detection and quantification of any nucleic acid modifying factors, including factors such as oxidant/antioxidant which are responsible for nucleic acid oxidative damage.

For the preparation of the formulation, conductive and electroactive particles are provided, which may be made of any suitable conductive material, such as carbon (graphite), gold and/or platinum or a mixture thereof. In general, the particles have the shape of flakes or balls, and exhibit a size of between 0.01 and 500 μm, preferably between 1 and 20 μm. Particles can also be mixed with or replaced by colloids, in which case the size ranges of from 0.01 and 1 μm.

If desired, the particles and/or colloids may be treated by physical or chemical means, such as laser or plasma irradiation, by mechanical grinding, laminating or heat (pyrolitic) treatment or with oxidizing, acidifying or bonding agents, such as e.g. ferrocene carboxylic acid, so as to make the sensor more selective, sensitive and to specify the dynamic range of the analysis when used as an electrochemical device.

Subsequent to the above optional pre-treatment, the nucleic acid material, i.e. DNA and/or RNA, is mixed with the particles obtained/provided as above either by evaporation or under reduced pressure from a solution containing said nucleic acids. In principle, the nucleic acid material may be any nucleic acid material of unspecific nature, such as salmon testes DNA, calf thymus DNA, or may exhibit specific sequences, such as provided by single stranded DNA (oligomers) and/or RNA which may later be hybridized to other nucleic acids, chemically linked to other substances, such as DNA dyes or DNA intercalants or solvated with the help of specific additives.

Together with the nucleic acid material, additives may be incorporated within the DNA molecules and/or may be co-mixed with the particles, such as e.g. intercalants (e.g. Ruthenium (2,2′-bipyridine)₃²⁺) which are used as electrochemical catalysts.

In a next step, the particles are mixed with the DNA such that a composite material is obtained wherein the conductive particles are embedded into the DNA.

The composite material is then either molded to any shape and without substrate or support materials or may be deposited onto a suitable substrate/support in thin or thick layers, in conventional ways, such as by printing, leading to a final sensor volume on which electrochemical, chemical and other reactions may be conducted. As a substrate/support, any non-conductive or also conductive material may be used, such as cellulose, polyester, polystyrene, metal, electrode, organic tissues.

The nucleic acid composite material is then dried at atmospheric or under reduced pressure at temperatures ranging from −230° C. to about 400° C. or more, with the use of pulsed air or not, during few seconds to several hours. All shapes of dried material may be obtained. The typical thickness of the dried layer ranges from 1 to several hundreds micrometers, preferably of from 5 to 50 μm. The temperature selected for drying is preferably in the range of 30° to 50° C., and the time period is from several seconds to several hours, preferably from about 1 minute to about 15 minutes.

Depending on the solvent added to the mixture and the drying condition, the resulting composite material can be porous or non-porous.

At the surface of the material thus obtained, particles, nucleic acids as well as optionally dye or other molecules are emerging from the resulting composite materials.

The material may be exposed directly to the substance to be analyzed and/or is available for further treatments. These treatments include mechanical polishing, light irradiation at any wavelength, UV, X-ray, photon treatments, other radioactive activations such as with alpha-, beta-particles or neutrons, chemical activation such as acidic or basic treatment, oxidation, electrochemical activation such as reduction, oxidation, biological, biochemical treatments or combination of these techniques.

All treatments may also be conducted at specific, geometrically well defined locations on the surface of the sensor. In addition, other insulating or conductive materials, such as polymeric solutions, metallic layers, inks, glues, solvents, etc. may be deposited onto certain locations/regions/areas on the surface. Thus, a patterning of the surface of the sensor may be obtained. The area of the sensor may be controlled by a first printing or molding stage, by the adding of layers geometrically defined that can be of different mixture compositions. Any step of printing, molding or treatment may be repeated in all kinds of sequences.

The sensor is connected as the working electrode into an electrochemical cell comprising a reference electrode such as a silver/silver chloride wire or layer and a counter electrode such as a platinum, gold, carbon wire or layer.

When using the sensor obtainable according to the above described method steps for determining a desired entity, the sensor is placed in contact with the sample to be analyzed, i.e. a sample suspected to modify and/or alter, e.g. oxidize the DNA material contained in the sensor. In general, the sample may be in any form allowing contact with the sensor, e.g. the form of a solution, a gas or even in solid form.

Depending on the nucleic acid modifying treatment, the electrochemical response of the sensor will be modified and/or altered. The electrochemical signal may be displaced in potential, in current, in impedance, in the quantity of charge transported across the sensor, or any combinations of these signals. Thus the electrochemical analysis can be performed by means of any coulometric, voltammetric, amperometric techniques or impedance analysis. In principle, an untreated sensor is used as reference and control purposes. Repeated electrochemical signals can be recorded over time and give an indication of the damaging effect dynamics.

The principle of the measurement, in case of nucleic acid oxidative damage, is the following: Reactive Oxygen Species (ROS) are generated via a Fenton reaction (Lloyd et al., Free Radical Biol. Med. 22(5) (1997), 885-8; H. J. H. Fenton, Proc. Chem. Soc. 9 (1983), 113), chemicals know to alter DNA such as styrene oxide, electrochemistry and/or any other nucleic acid altering treatment. Antioxidants are added to counteract the action of ROS, thereby allowing a measurement of their efficacy to neutralize the ROS.

Alternatively factors including pro- and/or anti-oxidants may be added to the solution. The presence and efficiency of these molecules as DNA damaging or protecting agents can then lead to a variation of the corresponding electrochemical signal.

This results in the possibility to characterize a given antioxidant, or a mixture of antioxidants in e.g. liquids, gel and gases. Applications include the analysis of any substance capable of holding antioxidant molecules including food, beverages, drugs, environments, liquids, gases, perfusion products, biological fluids such as saliva, blood, serum, plasma, urine tears, sweat, inter- and intra cellular fluids, etc.

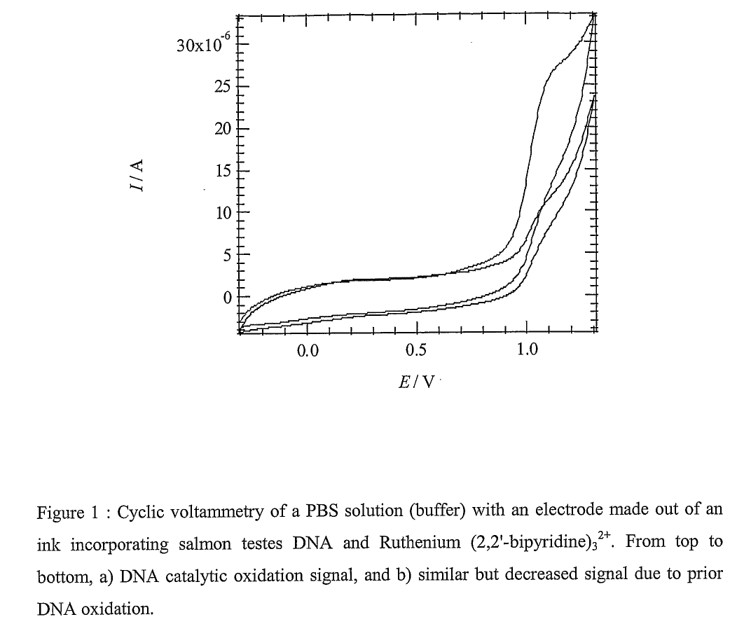

FIG. 1 shows the result of a cyclic voltammetry measurement with the disclosed invention of a) sensor made of unmodified composite materials containing Salmon testes DNA, used as reference blank, top curve and b) oxidized composite materials.

1. A method of obtaining a Nucleic acid composite material sensor comprising the steps of
(i) providing conductive particles;
(ii) mixing dry or wet nucleic acid material with the conductive particles thereby obtaining the nucleic acid composite material;
(iii) depositing the nucleic acid composite material onto a substrate or molding the nucleic acid composite material to be used as a working electrode; and
(iv) drying the nucleic acid composite material on the substrate thereby obtaining the nucleic acid material sensor

2. The method according to claim 1, wherein the conductive particles are made of carbon, gold, platinum, silver and/or colloids of the same materials.

3. The method according to claim 1, wherein the nucleic acid material is selected from the group consisting of double strand or single strand DNA or RNA of any lengths, ranging from a few nucleotides (oligomers) to several thousands of bases and synthetic nucleic acid of specific sequences.

4. The method according to claim 1, wherein the pre-treatment step (ii) comprises physical or chemical pretreatment of the particles with laser or plasma irradiation, mechanical grinding, laminating, heat, oxidizing, acidifying and bonding agents.

5. The method according to claim 1, further comprising pretreating the nucleic acids with DNA dyes, or intercalants.

6. (canceled)

7. The method according to claim 1, wherein the substrate consists of isolating material selected from the group consisting of paper, and polymers.

8. The method according to claim 1, wherein the substrate consists of conductive material selected from the group consisting of carbon ink, and metallic base.

9. The method nucleic acid composite material sensor according to claim 1, further comprising printing, layering, embedding or engraving said nucleic acid composite material onto said substrate.

10. The method according to claim 1, wherein the composite material is molded or injected with or without substrate onto a specific shape.

11. The method according to claim 1, wherein the nucleic acid composite material is deposited onto a carrier or molded or injected at a temperature ranging from −230° C. to 400° C.

12. The method according to claim 11, wherein the printed or molded or injected nucleic acid composite material is heat-treated at a temperature ranging from −230° C. to 400° C.

13. (canceled)

14. (canceled)

15. (canceled)

16. A method for detecting compounds or compositions having an oxidative or anti-oxidative activity, said method comprising:
providing a fluid comprising at least one compound or composition to be tested;
contacting said fluid with a nucleic acid composite material sensor according to claim 17;
subjecting the nucleic acid composite material sensor to conditions oxidative for nucleic acid molecules; and
determining an effect of the compound on the nucleic acid composite material sensor by measuring an electrochemical signal as compared to a reference, that had been subjected to the same conditions but had not been contacted with the compound.

17. A nucleic acid composite material sensor comprising
a nucleic acid composite material comprising
conductive particles; and
dry or wet nucleic acid material, and
a substrate.

18. The nucleic acid composite material sensor according to claim 17, wherein the conductive particles are made of carbon, gold, platinum, silver and/or colloids of the same materials.

19. The nucleic acid composite material sensor according to claim 17, wherein the nucleic acid consists of double strand or single strand DNA or RNA of any lengths, ranging from a few nucleotides (oligomers) to several thousands of bases.

20. The nucleic acid composite material sensor according to claim 19, wherein said DNA is bulk DNA, a synthetic nucleic acid and/or the mixture of the above.

21. The nucleic acid composite material sensor according to claim 20, wherein said bulk DNA is salmon sperm DNA or calf Thymus DNA, and said synthetic nucleic acid comprises poly (G), poly (A), poly (T), poly (U) or poly (C) sequences.

22. The nucleic acid composite material sensor according to claim 17, wherein the particles are pretreated with laser or plasma irradiation, mechanical grinding, laminating, heat, oxidizing, acidifying or with bonding agents.

23. The nucleic acid composite material sensor according to claim 22, wherein said bonding agent is ferrocene carboxylic acid.

24. The nucleic acid composite material sensor according to claim 17, wherein the nucleic acids are pretreated with DNA dyes, and/or intercalants.

25. The nucleic acid composite material sensor according to claim 24, wherein said intercalants are metal complexes comprising Ruthenium, ferro- and cobalt ions.

26. The nucleic acid composite material sensor according to claim 17, wherein the substrate consists of an isolating material selected from paper and polymers.

27. The nucleic acid composite material sensor according to claim 17, wherein the substrate consists of a conductive material selected from carbon ink, and metallic base.

28. The nucleic acid composite material sensor according to claim 17, wherein the nucleic acid composite material is printed, layered, embedded, or engraved onto said substrate.

29. The nucleic acid composite material sensor according to claim 17, wherein the nucleic acid composite material is molded or injected with or without substrate into a specific shape.

30. The nucleic acid composite material sensor according to claim 17, wherein the nucleic acid composite material is deposited onto the carrier or molded or injected at a temperature ranging from −230° C. to 400° C.

31. The nucleic acid composite material sensor according to claim 30, wherein the printed or molded or injected nucleic acid composite material is heat-treated at a temperature ranging from −230° C. to 400° C.

32. The method according to claim 3, wherein said DNA is bulk DNA, a synthetic nucleic acid and/or the mixture of the above.

33. The method according to claim 32, wherein said bulk DNA is salmon sperm DNA or calf Thymus DNA, and said synthetic nucleic acid comprises poly (G), poly (A), poly (T), poly (U) or poly (C) sequences.

34. The method according to claim 4, wherein said bonding agent is ferrocene carboxylic acid.

35. The method according to claim 5, wherein said intercalants are metal complexes comprising Ruthenium, ferro- and cobalt ions.
